(19) United States
(12) Patent Application Publication
(10) Pub. No.: US 2010/0049318 A1
(43) Pub. Date: Feb. 25, 2010

(54) Implantable Housing With Stabilizer

(75) Inventors: Claude Jolly, Innsbruck (AT); Stefan Nielsen, Innsbruck (AT); Dominik Hammerer, Innsbruck (AT); Martin Zimmerling, Patsch (AT); Gerhard Mark, Axams (AT)

Correspondence Address: Sunstein Kann Murphy & Timbers LLP, 125 SUMMER STREET, BOSTON, MA, 02110-1618, US

(73) Assignee: MED-EL ELEKTROMEDIZINISCHE GERAETE GMBH, Innsbruck (AT)

(21) Appl. No.: 12/545,274

(22) Filed: Aug. 21, 2009

Related U.S. Application Data
(60) Provisional application No. 61/090,758, filed on Aug. 21, 2008.
(60) Provisional application No. 61/102,984, filed on Oct. 6, 2008.

(51) Int. Cl. A61F 2/18

(52) U.S. Cl. 623/10

(57) ABSTRACT

Components of an implant system are described. An implant housing contains system components for performing system operating functions. An implant stabilizer extends out from the implant housing and implant data lead for interacting with an underlying curved bone surface to immobilize the implant housing in a fixed position.

The present application claims priority from U.S. Provisional Application 61/090,758, filed Aug. 21, 2008, and from U.S. Provisional Application 61/102,984, filed Oct. 6, 2008; which are incorporated herein by reference.

### FIELD OF THE INVENTION

The present invention relates to medical implants, and more specifically to cochlear implant systems.

### BACKGROUND ART

There are both long term and short term implantable devices that are positioned next to the skull. For example, the housing of a cochlear implant is typically placed on the temporal bone of the patient. Middle ear implants are also placed on the temporal bone. Other implants may be placed on the parietal or occipital bone. Pressure sensor devices and deep brain stimulator may also need to be placed on the skull. Usually a flat bed is drilled on the skull to form a flat footprint that receives the implant housing.

Implantable devices are tending to become larger as more and more system functionality is added. Moreover, more young patients are receiving implantable devices such as cochlear implants. As a result, the location where the implant is placed may involve greater curvature of the skull in all directions. And if the implant is not on a flat surface, it may have a tendency to rock, especially on a convex surface. Rocking and/or micro-movement of the implant due to poor immobilization may lead to infection and inflammation of tissues over time. Rocking can also cause wire breakage over time. The fragile implant data wiring must exit from the implant housing toward various other internal locations such as a middle ear transducer, cochlear implant electrode, deep brain electrode, visual cortex electrode, auditory brain stem electrode, inferior colliculus electrode etc.

Another issue is the development of thinner profile implant housings for pediatric patients. For a given set of electronic components, a thinner implant housing requires a larger surface having a greater footprint. This further increases the issues associated with the flat implant housing contacting the curved skull surface. Thus, the need for self-stabilization of the implant housing also increases. This is especially true for pediatric patients who have a relatively thin cortical bone in which drilling of a flat bed may not be possible beyond a depth of less than 1 mm.

So far these problems have been addressed by reducing the footprint of the implant housing, placing the implant housing on the flattest part of the skull, and drilling a flat bed to accommodate the flat undersurface of the implant housing. But as the size of the implant housing increases, the amount of drilling for the bed footprint also increases dramatically, and multiple sites may need to be drilled.

These problems have also been handled by placing the flat implant housing directly on the curved surface of the skull without drilling a bed. This can lead over time to wire breakage, skin inflammation, rocking of the implant housing on the skull underneath the skin, and the requirement to place of the implant housing in a flat region of the skull as much as possible. An unstable implant may furthermore be laterally displaced causing tissue damage and possible wire breakage.

### SUMMARY OF THE INVENTION

Embodiments of the present invention are directed to components of an implant system. An implant housing contains system components for performing system operating functions. An implant stabilizer extends out from the implant housing for interacting with an underlying curved bone surface to immobilize the implant housing in a fixed position.

In a more specific embodiment, the implant stabilizer may extend laterally away from the implant housing over the bone surface; for example, as one or more stabilizing wings made of polymer or silicone. In some embodiments, the implant stabilizer may be in the form of a mesh or grid, or made from a fabric material.

In some embodiments, the implant stabilizer may extend perpendicularly away from the implant housing onto the bone surface; for example, in the specific form of a compressible cushion adapted to conform to the bone surface. Or the implant stabilizer may include multiple positioning rods; for example, three. Some or all of the positioning rods may penetrate into the bone surface. The positioning rods may be made of a somewhat compressible polymer or metal. Or the implant stabilizer may be formed from a raised tread pattern for engaging the bone surface, such as from multiple pyramid shapes.

The implant stabilizer may be formed of a relatively flexible material that hardens over time. The implant stabilizer may be separable from the implant housing so that the implant housing can be removed without disturbing tissue around the implant stabilizer.

Embodiments of the present invention also are directed to components of an implant system where an implant data lead carries one or more system data signals, and an electrode stabilizer extends out from the data lead for interacting with an underlying curved bone surface to immobilize the data lead in a fixed position.

In a further such embodiment, the electrode stabilizer may extend laterally away from the implant data lead over the bone surface; for example, as a polymer or silicone stabilizing wing. The electrode stabilizer may be formed of a relatively flexible material that hardens over time. In addition or alternatively, the electrode stabilizer may be separable from the implant data lead so that the implant data lead can be removed without disturbing tissue around the electrode stabilizer.

### BRIEF DESCRIPTION OF THE DRAWINGS

FIG. 1 shows an example of an implant stabilizer in the form of lateral stabilizing wings.

FIG. 2A-C shows alternative examples of stabilizing wings.

FIG. 3A-B shows examples of implant stabilizers in the form of a conformable cushion underneath the implant housing.

FIG. 4A-B shows alternative examples of a stabilizing cushion.

FIG. 5A-C shows examples of implant stabilizers that extend in a perpendicular pattern from the implant housing.

FIG. 6A-C shows details of positioning rods that can be used as an implant stabilizer.

FIG. 7 shows an embodiment where the positioning rods penetrate into the underlying bone.

FIG. 8 shows an embodiment where the implant housing is detachable from the implant stabilizer.

FIG. 9 shows another embodiment where the implant housing is detachable from the implant stabilizer.

### DETAILED DESCRIPTION OF SPECIFIC EMBODIMENTS

Various embodiments of the present invention are directed to implantable components including an implant housing that contains system components for performing system operating functions. An implant stabilizer extends out from the implant housing for interacting with an underlying curved bone surface to immobilize the implant housing in a fixed position.

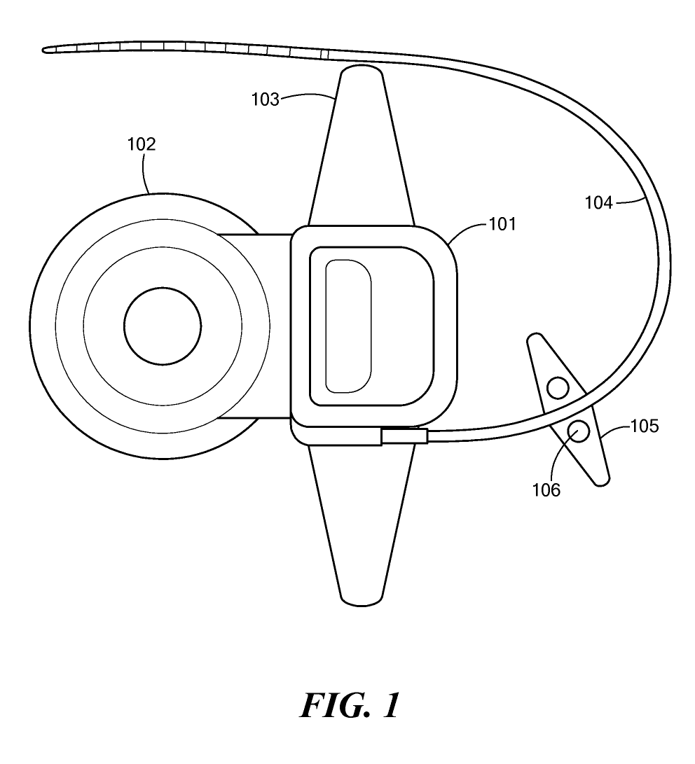

FIG. 1 shows an example of an implant stabilizer in the form of lateral stabilizing wings. A cochlear implant system includes an implant housing 101, receiver coil 102, and an implant data lead in the specific form of a stimulator electrode 104. An implant stabilizer is formed by a pair of polymer stabilizing wings 103 which extend laterally from the sides of the implant housing 101 for stabilizing the implant housing 101 in a desired fixed position next to the skull, under the skin and periosteum. This provides an improved interface between the flat rigid surface of the implant housing 101 and an underlying curved portion of the skull. This solution also avoids the need for drilling into the skull bone, and surgical time and complexity is considerably reduced, which is especially important for infant surgical procedures (both surgical blood loss reduction and curvature of the skull are much more pronounced in young children because of their small size). The embodiment shown in FIG. 1 also includes electrode stabilizing wings 105 made of silicone and extending from the sides of the stimulator electrode 104. The electrode stabilizing wings 105 also have fastener holes 106 through which a screw or other fastener may pass to immovably fix the electrode stabilizing wings 105 in proper position, for example, with respect to the mastoidectomy.

Stabilizing wings 103 and 105 are useful for stabilizing the implant structures since they can be placed under a periosteum pocket which is simply lifted from the skull surface. Very quickly after closure of the surgical incision, the stabilizing wings 103 and 105 are encapsulated by the healing tissue. The expanse of the stabilizing wings 103 and 105 around the implant housing 101 and stimulator electrode 104 respectively prevents undesired movement or migration of the implant structures.

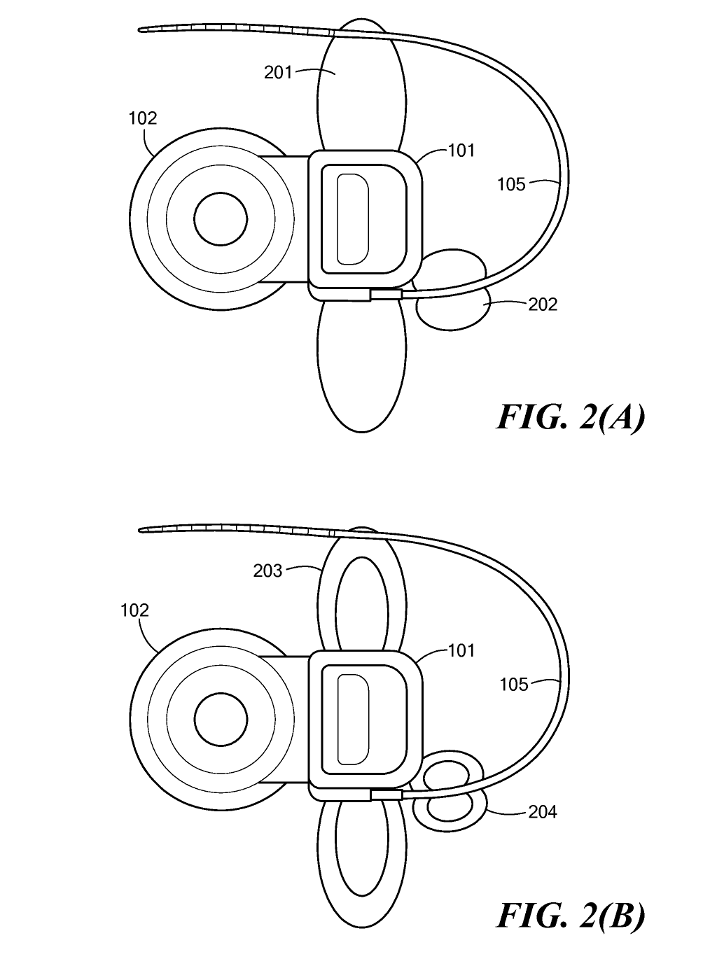

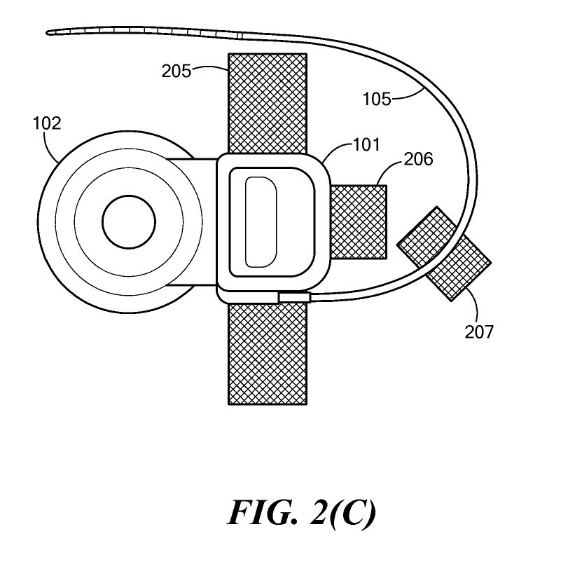

FIG. 2A-C shows some alternative embodiments of an implant stabilizer in the form of laterally extending wings. In FIG. 2A, the implant stabilizing wings 201 and electrode stabilizing wings 202 are more elliptical in shape, which may be useful in certain specific circumstances. The implant stabilizing wings 203 and electrode stabilizing wings 204 in FIG. 2B are similar in shape to the ones in FIG. 2A, but have the interior area of the wings shapes cut out to reduce the amount of contact surface with the nearby tissue. FIG. 2 C shows that rather than a polymer or silicone, the stabilizing wings 205 and 207 can be in the form of a mesh, grid, or fabric-like material. FIG. 2C also shows an embodiment having a secondary housing stabilizer 206 which provides additional stabilizing for the implant housing 101.

Embodiments such as the ones described above having laterally extending stabilizer wings may be adequate to stabilize and fix the implanted components in their correct positions in many circumstances, but in other specific circumstances the stabilizing action may not be sufficient to completely prevent rocking of the implant components on a convex surface such as the skull bone. Rocking and movement can be further reduced or eliminated with a compressible stabilizer cushion underneath the bottom surface of the implant housing.

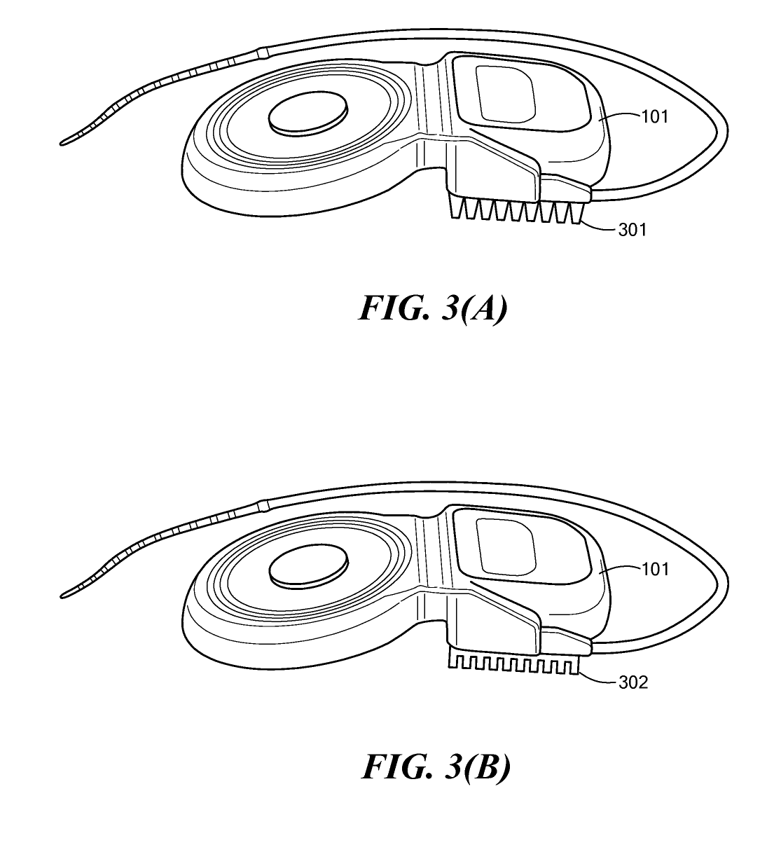

FIG. 3A-B shows embodiments of an implant housing 101 having such a stabilizer cushion. FIG. 3A shows a stabilizer cushion 301 having a pattern of compressible resilient cones (e.g., polymer or silicone) that adapt to the curvature of the underlying curved bone surface while providing support to the underside of the implant housing 101 to prevent it from moving. FIG. 2B shows another slightly different embodiment wherein the stabilizer cushion 202 is in the form of a pattern of cylindrical posts. Rather than an arrangement that is resilient or compressible, in some embodiments the stabilizing cushion may be made of a material that is initially soft when first implanted, but then hardens over time into a rigid structure that supports the underside of the implant housing 101 over the curved surface of the underlying bone. For example, body heat or moisture may accelerate or facilitate the hardening action.

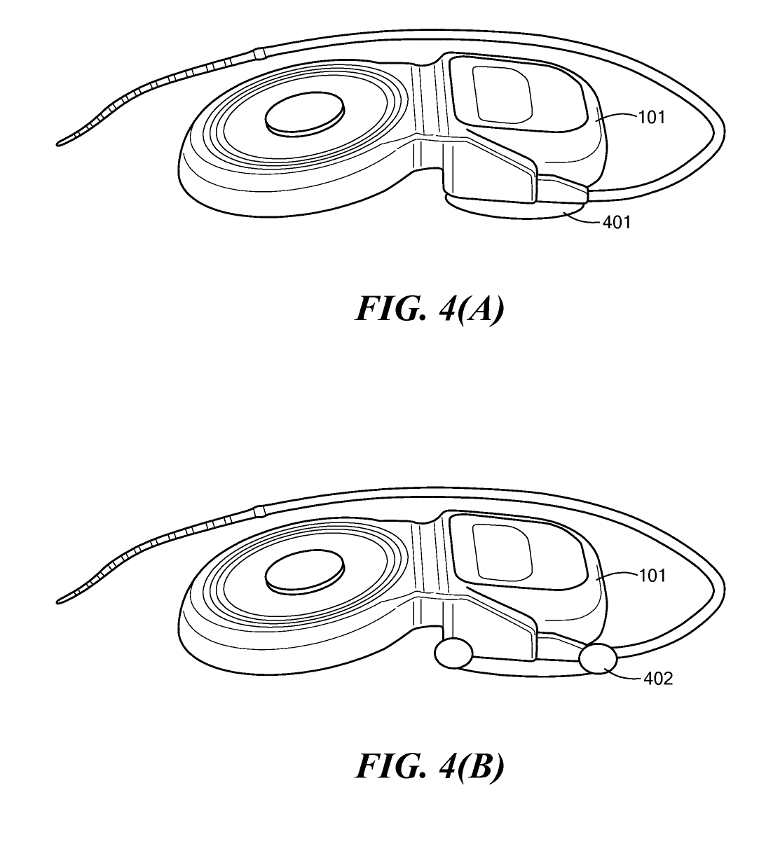

FIG. 4A-B shows other specific forms that a stabilizer cushion may take. In FIG. 4A, the stabilizer cushion 401 is a round pillow-shape attached to the underside of the implant housing 101. In FIG. 4B, the stabilizer cushion 402 has a donut-like shape that follows the contour and edges of the implant housing 101, which rest on and are supported and stabilized by the outer donut of the stabilizing cushion 402. Such a stabilizing cushion may be resilient and compressible to continuously support the implant housing 101, or initially soft when first implanted and then harden over time into a rigid structure. Some embodiments may combine a stabilizing cushion arrangement with a lateral stabilizing wing arrangement as described above for additional stability. In some embodiments, a stabilizing cushion may be a separable structure from the implant housing.

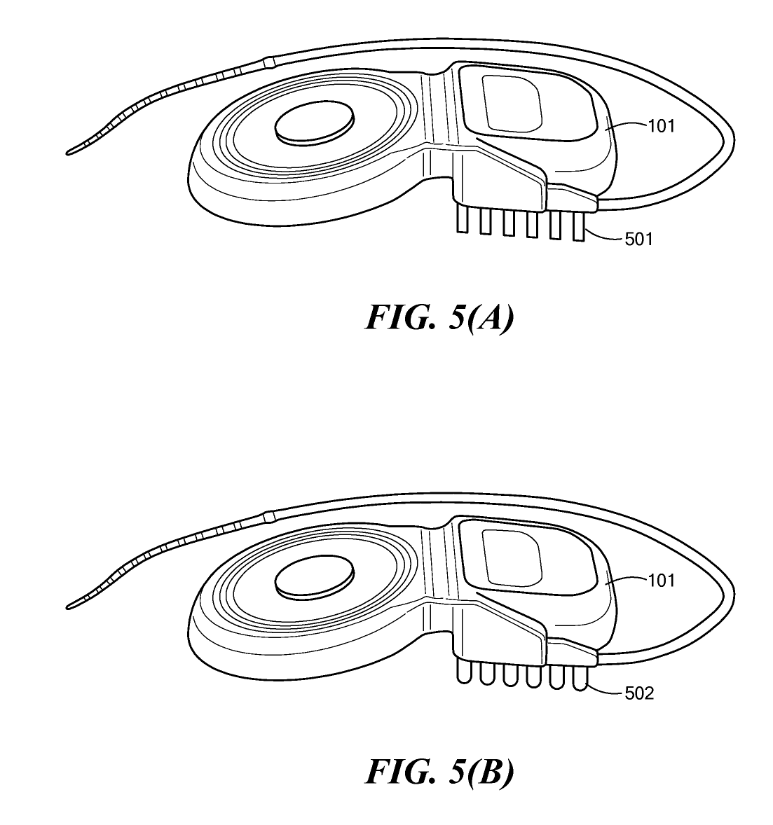

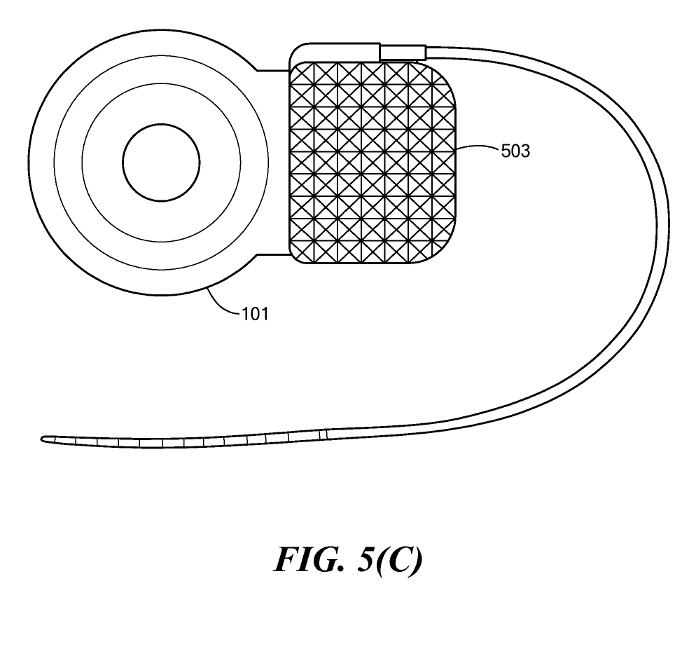

FIG. 5A-B shows examples of embodiments where the stabilizer cushion 501 and 502 may be more in the form of a number of thin rods or eggs that act as collapsible teeth as in a brush. FIG. 5C shows an embodiment wherein the stabilizer cushion 503 is in the form of a tread that engages the surface of the curved bone beneath, for example, a tread based on multiple pyramid shapes as shown. The tread pattern engages the underlying bone surface to prevent slipping movement of the implant housing 101 over the surface, much like a tire tread prevents slipping of a tire on the road.

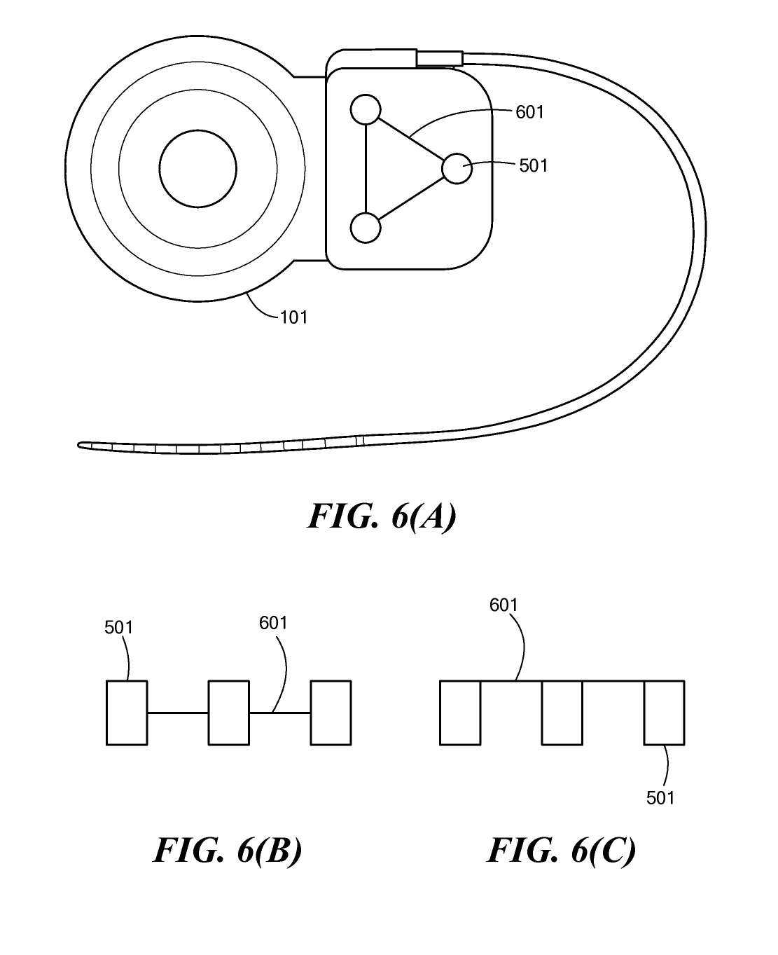

Another embodiment may have just a few compressible polymer rods distributed at key locations underneath the surface of the implant housing as shown in FIG. 6A. In FIG. 6A, the underside of implant housing 101 has a triangular arrangement of three stabilizing posts 501 connected by a flexible or rigid connector wire 601. This embodiment has the advantage that all the stabilizing posts 501 are in contact with the underlying skull and the implant housing 101 will not rock, tilt, or move.

The distribution, size and heights of the stabilizing posts 501 can be optimized to provide a useful cushion for support and stabilizing after some compression without compromising the thickness of the implant housing 101. The stabilizing posts 501, for example, may be only distributed at the edges of the implant housing 101 and provide a support for the edges of the implant housing 101 if it sits on a convex or tilted bone surface. The polymer material of the stabilizing posts 501 may be resiliently compressible, or may harden and rigidify over time. In some embodiments, the stabilizing posts 501 may be separable from the implant housing 101 so that the stabilizing posts 501 may be pre-attached to the bone surface 702 and then the implant housing 101 fitted over them. Such an arrangement would also facilitate later removal of the implant housing 101 for repair or replacement without disturbing the bone or tissue at the site of the stabilizing structure. And while the foregoing describes a specific embodiment based on the use of three stabilizing posts 501, the idea can be formulated more generally in the form of some number N posts: 3, 4, 5, etc. which may vary in specific embodiments.

As shown in FIG. 6B, the connector wire 601 may connect to the center of the stabilizing posts 501, which holds them together rather tightly to prevent their spreading. Or as shown in FIG. 6C, in some applications it may be useful to have the connector wire 601 up higher on the stabilizing posts 501 to allow more spreading of the stabilizing posts 501 to better accommodate the implant location geometry. The stabilizing posts 501 and connector wire 601 may be made of metallic or polymer material.

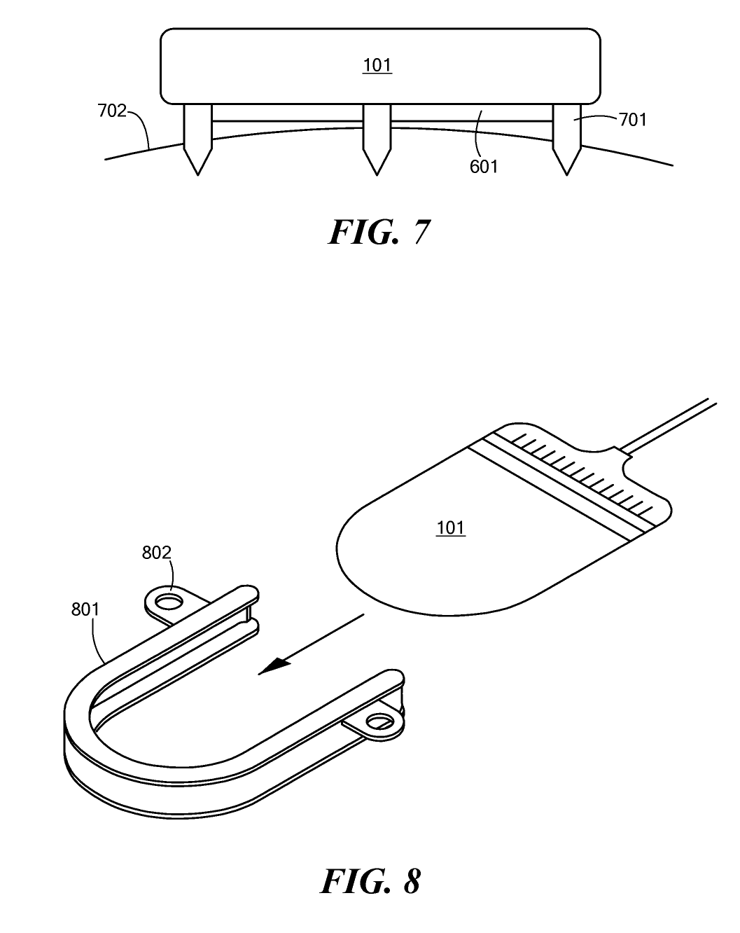

FIG. 7 shows an example of an embodiment where a triangular arrangement of hard penetrating stabilizer spikes 701 and lateral connecting wire 601 stabilize the implant housing 101 over a curved bone surface 702. The stabilizer spikes 701 may be of the same material as the implant housing 101 (e.g., titanium, titanium alloy, or ceramic). The stabilizing spikes 701 are designed and arranged to slightly penetrate into the bone surface 702. In some embodiments, some of the stabilizing spikes 701 may penetrate into the bone surface 702, while others do not. And again, while the use of three stabilizing spikes 701 is described, the idea can be formulated more generally as some number N spikes: 3, 4, 5, etc. which may vary in specific embodiments.

The height of the stabilizing spikes 701 should be such that even for a highly curved infant skull, the implant housing 101 is supported and stabilized in a fixed position. In such circumstances, the position and elevation of the stabilizing spikes 701 out from the bottom surface of the implant housing 101 controls the extent of their penetration into the bone surface 702. With infant skulls, the amount of penetration into the relatively thin skull bone will be limited by contact of the bottom surface of the implant housing 101 with the bone surface 702. For a relatively high bone curvature, as in young children with a small head, the stabilizing spikes 701 would only minimally penetrate into the bone surface 702 when the bottom surface of the implant housing 101 is in contact with the bone surface 702. For less bone curvature, as in adults, the stabilizer spikes 701 would penetrate more deeply into the bone surface 702. This does not pose a problem because of the greater thickness of the bone surface 702 underneath the implant housing 101, provided an appropriate shape and length of the stabilizing spikes 701. In some embodiments, the stabilizing spikes 701 may be separable from the implant housing 101 so that the stabilizing spikes 701 may be pre-attached to the bone surface 702 and then the implant housing 101 fitted over them.

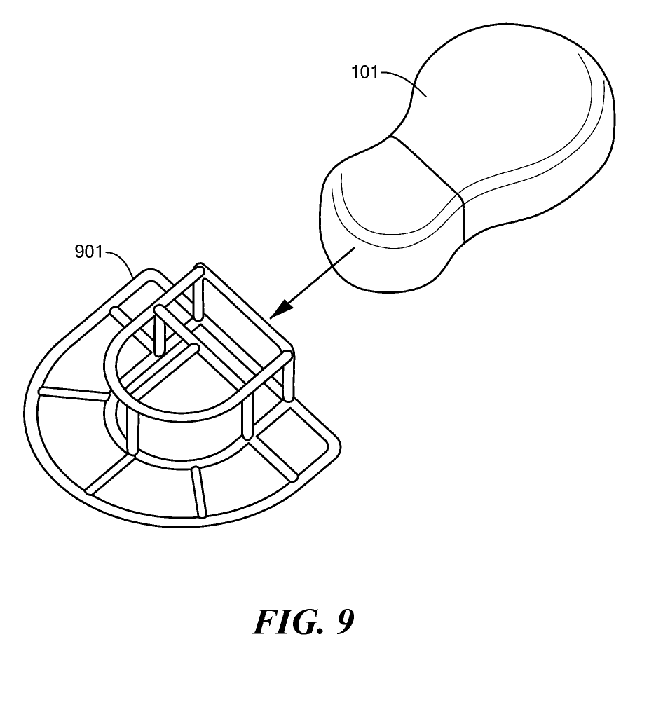

FIG. 8 shows another embodiment where a separate polymer implant stabilizer 801 may be pre-placed on the bone surface and attached by one or more anchoring flanges 802. After the implant stabilizer 801 has been installed, the implant housing 101 can be slid snugly into position. This arrangement allows for easy removal and replacement of the implant housing 101 without disrupting the polymer surface of the implant stabilizer 801 or the surrounding bone and tissue that has consolidated over time. The old implant housing 101 is simply pulled out, and a new one fit back into the existing site which stays in place and has not moved. FIG. 9 shows another embodiment of a bird cage-like implant stabilizer 901 that is pre-placed on the bone site and into which the implant housing 101 slides.

Embodiments of the present invention allow implanted components to be stabilized in a fixed position on a non-uniform or tilted bone surface such as a curved skull without having to drill a flat bed adapted to the shape of the implant housing. Surgical time and risk also are reduced, and long-term stability of the implanted components is improved by preventing or minimizing movement of the implant data lead. Moreover, as implant housings continue to get thinner but larger, the implant stabilizer removes or reduces the need for extensive drilling to make a flat surface bed to receive the implant housing.

Although various exemplary embodiments of the invention have been disclosed, it should be apparent to those skilled in the art that various changes and modifications can be made which will achieve some of the advantages of the invention without departing from the true scope of the invention.

What is claimed is:

1. An implantable apparatus for an implant system comprising:
an implant housing containing system components for performing one or more system operating functions; and
an implant stabilizer extending out from the implant housing for interacting with an underlying curved bone surface to immobilize the implant housing in a fixed position.

2. An apparatus according to claim 1, wherein the implant stabilizer extends laterally away from the implant housing over the bone surface.

3. An apparatus according to claim 2, wherein the implant stabilizer is a stabilizing wing.

4. An apparatus according to claim 3, wherein the stabilizing wing is formed of a polymer material.

5. An apparatus according to claim 3, wherein the stabilizing wing is formed of a silicone material.

6. An apparatus according to claim 2, wherein the implant stabilizer is in the form of a mesh or grid.

7. An apparatus according to claim 2, wherein the implant stabilizer is formed from a fabric material.

8. An apparatus according to claim 1, wherein the implant stabilizer extends perpendicularly away from the implant housing onto the bone surface.

9. An apparatus according to claim 8, wherein the implant stabilizer is a compressible cushion adapted to conform to the bone surface.

10. An apparatus according to claim 8, wherein the implant stabilizer includes a plurality of positioning rods.

11. An apparatus according to claim 10, wherein at least one of the positioning rods penetrates into the bone surface.

12. An apparatus according to claim 11, wherein all the positioning rods penetrate into the bone surface.

13. An apparatus according to claim 10, wherein there are three positioning rods.

14. An apparatus according to claim 10, wherein the positioning rods are compressible.

15. An apparatus according to claim 10, wherein the positioning rods are formed of a polymer material.

16. An apparatus according to claim 8, wherein the implant stabilizer includes a raised tread pattern for engaging the bone surface.

17. An apparatus according to claim 16, wherein the tread pattern includes a plurality of pyramid shapes.

18. An apparatus according to claim 1, wherein the implant stabilizer is formed of a relatively flexible material that hardens over time.

19. An apparatus according to claim 1, wherein the implant stabilizer is separable from the implant housing so that the implant housing can be removed without disturbing tissue around the implant stabilizer.

20. An implantable apparatus for an implant system comprising:
an implant data lead for carrying one or more system data signals; and
an electrode stabilizer extending out from the data lead for interacting with an underlying curved bone surface to immobilize the data lead in a fixed position.

21. An apparatus according to claim 20, wherein the electrode stabilizer extends laterally away from the implant data lead over the bone surface.

22. An apparatus according to claim 21, wherein the electrode stabilizer is a stabilizing wing.

23. An apparatus according to claim 22, wherein the stabilizing wing is formed of a polymer material.

24. An apparatus according to claim 20, wherein the electrode stabilizer is formed of a relatively flexible material that hardens over time.

25. An apparatus according to claim 20, wherein the electrode stabilizer is separable from the implant data lead so that the implant data lead can be removed without disturbing tissue around the implant stabilizer.
